(19) United States
(12) Patent Application Publication
(10) Pub. No.: US 2025/0160986 A1
(43) Pub. Date: May 22, 2025

(54) MEDICAL IMAGING GUIDED MAGNETIC ACTUATION AND NAVIGATION SYSTEM FOR CLINICAL APPLICATIONS

(71) Applicant: MULTI-SCALE MEDICAL ROBOTICS CENTER LIMITED, Hong Kong (CN)

(72) Inventors: Moqiu ZHANG, Hong Kong (CN); Lidong YANG, Hong Kong (CN); Li ZHANG, Hong Kong (CN)

(21) Appl. No.: 18/726,834

(22) PCT Filed: Dec. 23, 2022
(86) PCT No.: PCT/IB2022/062717

§ 371 Date: Jul. 5, 2024

Related U.S. Application Data
(60) Provisional application No. 63/297,661, filed on Jan. 7, 2022.

(51) Int. Cl. A61B 34/00; A61B 34/30; A61B 90/00

(52) U.S. Cl. CPC A61B 34/73; A61B 34/30; A61B 90/37; A61B 2034/304

(57) ABSTRACT

This invention provides a device for tracking of a magnetic element and a system comprising said device; said device comprises: a) a magnetic actuation unit, comprising: i) an eye-in-hand sensing module; ii) a plurality of magnetic sources arranged evenly around said eye-in-hand sensing module, each of said plurality of magnetic sources is tilted at a tilt angle and separated from an adjacent magnetic source at an adjacent angle; and iii) an adjustment mechanism connected to each of said plurality of magnetic sources for adjusting said tilt angle and said adjacent angle to achieve a desired magnetic field; b) a robotic platform for mounting of said magnetic actuation unit and providing dexterous pose control of said magnetic actuation unit.

### FIELD OF THE INVENTION

The present invention relates to an integrated system combining a mobile magnetic actuation unit and imaging tools for remote control of a magnetic robot through a body. The mobility of the robotic platform ensures a sufficient workspace for magnetic actuation and target tracking for clinical applications.

### BACKGROUND OF THE INVENTION

Magnetic robotics shows excellent potential in minimally invasive medical applications. Because of the intrinsic merits of the magnetic field, such as less distortion when penetrating the human body; harmless to biological tissue, and efficient wireless power transmission, medical magnetic robots have been applied to various clinical tasks, including but not limited to tissue sampling, drug delivery; real-time diagnosis. Magnetic manipulation systems that perform multiple degree-of-freedom magnetic control have been proposed to realize dexterous magnetic manipulation. Magnetic manipulation systems can be classified into two categories by the magnetic source used: Permanent Magnetic System (PMS) and Electromagnetic System (EMS).

For the first category, a robotic platform is commonly utilized to control the permanent magnet actuator's pose to generate the desired magnetic field, a safe and effective method for actuating various magnetic robots. However, such systems are hard to create complex or fast-changing modulated magnetic fields. Furthermore, the fast degrade magnetic field limits adequate actuation depth, and the magnetic field is hard to turn off. The desired magnetic field is controlled by changing the supplied current for EMS. Using several electromagnets arranged around the workspace, one could realize dexterous magnetic manipulation ranging from micrometer robots to centimeter robots. The supplied currents control the magnetic field, and a stronger magnetic field would lead to difficulties in temperature control and risk control. The workspace is often surrounded/half-surrounded by the electromagnets, limiting the technology to benchtop experiments. EMS can generate a more controllable magnetic field, but the footprint is significant and needs supporting subsystems such as temperature monitoring & cooling unit and the current control unit. The magnetic field estimation is also more challenging due to the soft iron core's nonlinear behavior. The proposed invention includes a mobile electromagnetic actuation unit that employs a robotic platform that could bring electromagnets to a large workspace and avoid interference with the imaging tools. Dexterities of the system are determined by the number of electromagnets and the controllable DOF of the robotic platform. For different magnetic field generation requirements, the flexibility of the magnetic actuation unit can be designed accordingly.

To bridge the gap between the benchtop test and the clinical applications, various imaging and tracking methods are developed to conduct in-vivo magnetic robots actuation and navigation. Visual servoing by cameras or stereoscopes is often used in a laboratory environment and is difficult to be applied as a tracking tool in clinical applications. Alternative localization and imaging methods are needed for realistic scenarios where the working environment does not allow the line of sight. X-ray is one commonly used medical imaging method that could capture the information inside the human body with sufficient accuracy and refresh rate. Ultrasound imaging is another effective and efficient imaging tool that could acquire the section view of the imaging environment with micrometer level accuracy. Magnetic localization is a tracking method that could extract a magnetic robot's absolute position and orientation according to the magnetic field generated by the embedded magnets. In the proposed invention, one or more medical imaging tools can be integrated into the imaging and tracking unit for precise and accurate manipulation. Multiple imaging and tracking fusion methods are embedded in the host control unit for different imaging modalities.

Medical robots' actuation and navigation methods include hydraulic, tendon driven, and magnetic driven. Hydraulic actuation can be applied to medical robots to generate active deformation and locomotion. Due to the integration with tendons, medical catheter robots could realize functions such as steering, buckling, and stiffness regulation. Due to the intrinsic advantages of the magnetic field, such as being biological friendly and less distortion when passing through low permeability structure, magnetic actuation is widely applied to wireless control of untethered medical robots for in vivo applications. In the present invention, the actuation magnetic field is generated by the mobile magnetic source, which has a reconfiguration design. It could fulfill the requirement of various field generation methods by adjusting the relative position of each source or increasing the total source number.

Magnetic medical robots can be classified into tethered medical robots and untethered medical robots. Catherization is one of the widely adopted methods applied to medical practice, such as cardiac ablation, tissue sampling, cell/drug delivery, and treatment of vascular disease therapy. Tethered capsule robots are designed for GI tract inspections and real-time diagnosis. Alternatively, untethered robots are another type of controlled object that could better adapt to the tortuous environment and perform medical tasks in the hard-to-reach region of the human body. Remote wireless control of the untethered robot requires a more robust control algorithm and precise imaging tracking method. Moreover, the robots' advanced and complex locomotion principle put a higher-level requirement of field generation such that stronger and more complicated fields are needed. Untethered robots are widely used in endovascular applications or GI tact therapy, where standard medical tools are hard to access non-invasively. Targeted different medical applications, magnetic robots are designed with multiple functions and require different control field requirements. Magnetic force-driven robots have been studied to accomplish GI tract monitoring or microsurgery in the eyes. Torque actuated robots such as helical robots or rolling robots would have more efficient locomotion and smaller scale. Furthermore, some functional designs of the robots, such as drug delivery or tissue sampling, need a specific magnetic field design to trigger the mechanism accurately. The proposed system would generate a complex controllable field in a large workspace with an optimized configuration. The combination of dexterous magnetic actuation and real-time pose and environment information can be applied to clinical applications such as long-distance drug delivery, blood clot clearance, whole GI tract monitoring, and therapeutic procedure in the distal endovascular system.

U.S. Pat. No. 7,311,107 B2 relates to a device and the control system for actuating and localizing an in vivo vehicle by detecting and manipulating the strength and direction of the electromagnetic field vector of the car. It is mentioned that soft magnetic cores can be mounted in electromagnets.

Another patent, numbered U.S. Pat. No. 6,311,082 B1, discloses a movable bed system for supporting the patient and sets of electromagnets mounted on a curved shell below the said bed, which can provide magnetic fields or gradients through proper energization of subsets of the magnets.

U.S. Pat. No. 6,148,823 relates to a system for controlling magnetic elements in the body using a gapped toroid magnet, which comprises a pair of mutually attracting opposed magnets separated by a gap. Said magnets are preferably permanent magnets connected by a flux return path. It also discloses that the magnets can be mounted on a movable support, and the system has portable support for the patient and an x-ray imagery device.

U.S. Pat. No. 6,216,026 B1 relates to an invention with a controllable magnetic moment suitable for navigating a catheter or a flexible endoscope within a patient's body when incorporated with MRI. The disclosed embodiment has coils located in three different axis directions, thus providing various combinations of magnetic moments by changing current input in three coils.

U.S. Pat. No. 8,830,648 B2 discloses a magnetic actuation system, Octomag, a magnetic system with two sets of electromagnetic coils. Each group comprises four electromagnets or permanent magnets, which can control a millimeter-scale robot to move with 5 degrees of freedom within a small workspace, like a cavity for human eyes.

A further device is disclosed in U.S. Pat. No. 8,041,411 B2. The said patent discloses a magnetic control system with circumferentially movable magnets mounted on a curved arm and utilizing images from the x-ray to navigate the magnetic element.

An apparatus consisting of 8 magnetic coils with magnetic cores mounted on two opposed spherical shells are introduced in U.S. Pat. No. 8,027,714 B2. Said embodiment allows servo motor to position the relative distance between the two said sets of electromagnets and introduced radar for imagery to eliminate radiation such as X-rays. Said embodiment can generate field strength and gradient between B=0.04 T˜0.15 T and 1.6 T/m to 3.0 T/m, respectively, thus exerting sufficient strength and orientation to move a magnetically responsive catheter tip.

### SUMMARY OF THE INVENTION

This invention provides a device for tracking of a magnetic element. In one embodiment, said device comprises: a) a magnetic actuation unit, comprising: i) an eye-in-hand sensing module: ii) a plurality of magnetic sources arranged evenly around said eye-in-hand sensing module, each of said plurality of magnetic sources is tilted at a tilt angle and separated from an adjacent magnetic source at an adjacent angle; and iii) an adjustment mechanism connected to each of said plurality of magnetic sources for adjusting said tilt angle and said adjacent angle to achieve a desired magnetic field: b) a robotic platform for mounting of said magnetic actuation unit and providing dexterous pose control of said magnetic actuation unit.

This invention also provides a system for tracking of a magnetic element. In one embodiment, said system comprises: a) a magnetic element: b) one or more devices of claim 1: c) an eye-to-hand imaging system (2); and d) a computer processor: wherein said computer processor executes an algorithm for collaborating said one or more devices and said eye-to-hand imaging system (2), said algorithm comprises the steps of: i) receiving pose information of components in said system: ii) calculating desired trajectories of said one or more devices of claim 1 and said eye-to-hand imaging system (2) based on a command for manipulating said magnetic element from a user before an operation: iii) analyzing real time positions of objects in field-of-view of said eye-to-hand imaging system during said operation; and iv) adjusting said desired trajectories based on said real-time positions using a predictive control strategy to detect potential collision.

### BRIEF DESCRIPTION OF THE FIGURES

FIG. 1 shows a schematic diagram of the integrated system for magnetic control of a magnetic element in a patient's body.

FIG. 2 shows the workflow diagram of the integrated system.

FIG. 3(a) shows one embodiment of the electromagnetic source design showing the adjacent angle.

FIG. 3(b) shows one embodiment of the electromagnetic source design showing the tilt angle.

FIG. 3(c) shows the embodiment of the invention that shows the reconfigurable coils support structure design for an individual coil.

FIG. 3(d) shows one embodiment of the invention that could adjust the pose of the individual electromagnet by one rotational actuator.

FIG. 3(e) shows one embodiment of the invented pose adjustment device that is composed of a linear actuator 28, a universal ball joint 29, and a rotational joint 30.

FIG. 3(f) shows the embodiment of the invented pose adjusting design that uses a single linear actuator to adjust the pose of the individual electromagnetic coil.

FIG. 3(g) shows four embodiments of the invented core tip design.

FIG. 3(h) shows two embodiments of the invented electromagnetic coils shape design.

FIG. 4(a) shows the cross-section of one embodiment of the electromagnetic coils cooling design.

FIG. 4(b) shows one embodiment of the cooling channel design.

FIGS. 4(c) to 4(f) show four embodiments of the invented core cooling system.

FIG. 5(a) shows a serial robotic arm is designed to realize the motion control by using two rotational joints 36, 37 to control the in-plane motion and other two rotational joints to control the out-of-plane motion.

FIG. 5(b) shows a robotic platform that is designed in a leverage structure for sufficient loading tolerance that could support a larger magnetic source.

FIG. 5(c) shows one embodiment of the invented robotic platform structure.

FIG. 5(d) shows the embodiment of the invented design that uses mobile magnetic sources to realize multiple points of magnetic field control.

FIG. 6(a) shows an embodiment of the eye-in-hand tracking system design whereby the ultrasound probe is held by a micro-Stewart Platform that could control 6 DOF of the US imaging plane.

FIG. 6(b) shows an embodiment of the eye-in-hand tracking system design whereby the one rotational DOF is controlled by a rotational actuator.

FIGS. 7(a) to 7(c) shows embodiments of the magnetic isolation shield design 50 and the magnetic field monitoring system design 51.

FIG. 8(a) shows the arrangement of the subsystems such that the magnetic actuation and imaging tools share the same human-size workspace. The subsystems sit on rails 58, 54 that allow stable ground motion around the patient bed.

FIG. 8(b) shows the details of the magnetic source design from the invention that consists of 4 coils and an eye-in-hand ultrasound imaging tool 59.

### DETAILED DESCRIPTION OF THE INVENTION

This invention provides an integrated magnetic system for actuation and navigation of magnetic elements through a body which consists of a mobile magnetic source for generating controllable magnetic field in a large workspace, an imaging and tracking system for localizing the target and acquiring the internal information of the body, a central process system for implementing control algorithm and data analysis, a human machine interface to translate operators' orders to control signals and a medical device equipped supporting bed that could reflect the patient's condition. The magnetic source is controlled by a robotic platform that allows heavy load magnetic source movement in a human-scale workspace. The desired magnetic field is generated by the collaboration of the robotic platform and the supplied current. The motion control of the imaging and tracking system allows a human-scale tracking space same as the magnetic actuation system and consists of multiple imaging modalities and tracking tools.

Based on the prior work, one object of the invention is an integrated system combining magnetic field actuation and medical imaging tools for extensive workspace manipulation and navigation of medical magnetic robots for in vivo clinical applications. Desired magnetic field generation in a large workspace is achieved by collaborating the robotic platform and the magnetic source. In order to satisfy the requirement of clinical applications, imaging and tracking subsystem are integrated to localize the controlled objects and image the body's internal information.

One feature of the present invention is that mobility ensures the successful collaboration of the magnetic actuation and imaging modality. For different medical applications, the present invention could adjust accordingly to generate specific control fields without interfering the imaging and tracking quality. Furthermore, the magnetic source can move to the vicinity of the controlled object to generate desired actuation field such that it could avoid current overshoot and soft iron core saturation. The present invention has a human-scale workspace and can also focus on the specific region without using a bulky magnetic source.

A cluster of reconfigurable magnetic sources generates the magnetic field. In one embodiment, the magnetic source is made of a permanent magnet. A pose control device generates the desired magnetic field that controls the magnets' orientation. Based on the shape of the permanent magnet and the desired actuation field, the positioning device could be designed accordingly. Actuation fields such as a rotating field or oscillating field can be realized by regulating the moving pattern of each magnetic source. In one embodiment, the magnetic sources are electromagnetic coils. The desired magnetic field could be generated by the collaboration of the robotic platform and the supplied current. According to the imposition principle, the magnetic field at the desired position is the summation of the magnetic field of each magnetic source. By changing the relative position of each electromagnet, specific fields include but are not limited to gradient-dominant field, torque-dominant field, gradient-free field and torque-free field can be achieved.

The electromagnetic source assembly has a reconfigurable design to accommodate the relative configuration adjusting device. In one embodiment, the magnetic source is composed of several permanent magnets that could rotate and translate to generate the desired magnetic field. In one embodiment, the magnetic source is composed of a cluster of electromagnetic coils. The supplied current, the orientation, and the relative position can be adjusted to the generated desired field. In one embodiment, the core of the magnetic coil is made of soft iron with the internal cooling channel. The tip shape is designed to satisfy different magnetic field generation requirements.

In one embodiment, a temperature control subsystem includes a cooling module, temperature sensing module and circulating module is invented to protect the electromagnetic source. In one embodiment, the cooling module consists of external cooling that the cooling device is at the coil's surface. In one embodiment, the internal cooling design is implemented on the core of the electromagnetic core. The internal temperature can be better controlled to design a cooling channel inside the soft iron core. The design of the internal cooling structure, which is designed by finite element analysis, is deployed when the external cooling unit cannot provide sufficient temperature control. A numerical method to model the magnetic field generated is invented to overcome the nonlinearity brought by the internal cooling coil. The six-point temperature sensing deployment method is invented to provide better knowledge of the real-time condition of the coil. In one embodiment, the coolant circulating speed and the cooling temperature is regulated according to the feedback sensing temperature.

For electromagnetic sources, a spatial arrangement method of the electromagnetic coil is invented to generate the desired magnetic field without compromising the accessibility and compatibility. In one embodiment, the coils are arranged symmetrically with axes coinciding at one point such that the focus point could have the largest field strength. In one embodiment, the multiple focuses are designed to allow multiple local maximum fields. In one embodiment, the coils are arranged to generate a uniform strength field. In one embodiment, the coils are arranged to generate a uniform-gradient field.

A current regulating device is invented to provide a large four-quadrant power supply to the electromagnetic coil. The current regulating subsystem consists of a bi-directional DC power supply, an H-bridge circuit supported by an H-bridge driver circuit and a microcontroller for generating high-frequency control PWM signals.

A magnetic actuation system configuration design method is invented to avoid collision with patient bed and interference to the imaging & tracking system. Based on the invented method, the workspace is scalable for different experiment objects and compatible with doctors' inspections.

In one embodiment, the bedside placement configuration is designed. It is compatible with the C-arm imaging module and allows enough access space for physician inspections. In one embodiment, the magnetic actuation system is installed on a U shape sliding rail for better adjustment to the operating room environment. In one embodiment, multiple sets of the mobile magnetic source are designed to either focus on multiple points applications or optimize field generation at one point.

A robotic platform supported mobile magnetic source is invented to provide numerous degrees of freedom (DOF) for magnetic field generation. The mobility can be realized in different forms. In one embodiment, a serial robotic arm supports the magnetic source, which has less footprint but stricter load requirement. In one embodiment, a three-DOF robotic structure made of screw tables is invented to increase the load and precision. In one embodiment, a side-mounting parallel robotic structure is used to adjust the pose of the magnetic source. The mobility can be realized in different forms.

A hybrid medical imaging system is invented to fuse different types of tracking and imaging modules. In one embodiment, an eye-to-hand imaging system integrated with a mobile C-Arm is designed to realize large workspace-controlled target tracking. In one embodiment, the eye-in-hand tracking system is integrated with the mobile magnetic source. Such design ensures an accurate tracking performance in a large workspace without complicated structural design or extra sensing support module. In one embodiment, an eye-to-hand sensing system and an eye-in-hand sensing system are combined to provide information for in-body magnetic robot navigation.

A magnetic field hybrid modeling method is invented to estimate the magnetic field in the workspace for various electromagnetic coil designs. Prior inventions present the technique of modeling magnetic field by dipole model, variant dipole model and analytical eddy current based model. The present modeling method uses a learning-based mapping approach to combine FEM data and calibrate data to estimate the generated field for different shape designs and core designs.

A magnetic field prediction method for movable magnetic sources is invented such that the period of transition between each position is taken into consideration. Implementing the invented method makes the magnetic field affected on the controlled object continuous. One challenge of the invented magnetic actuation system is accurately predicting the magnetic field.

The invented integrated system combines mobile magnetic sources and medical imaging tools to provide precise magnetic navigation and actuation for clinical applications. The magnetic system could move with the controlled target and navigate the magnetic elements through a long-distance without using large current or superconductive material. The imaging system is collaborated with the magnetic source to provide real-time controlled targets' pose and the internal body information. A multi-functional patient bed is designed to provide connections to medical devices. In one embodiment, a sensing and tracking module is embedded in the patient bed to provide localization of the controlled object. In one embodiment, the patient bed is integrated with ECG and ventilators.

This invention provides an integrated navigation system that combines mobile magnetic actuation and medical imaging tools for manipulation of a magnetic element for in vivo applications. In one embodiment, said system comprises: (a) a mobile magnetic source that is controlled by a robotic platform to realize large workspace magnetic field generation in the body of the patient with interference with the imaging system: (b) an imaging system for providing the position of the controlled element and the internal body information in a human-scale workspace, wherein the eye-in-hand sensing module and eye-to-hand imaging system work together to provide accurate and precise localization for control and diagnosis purpose, wherein the eye-in-hand sensing module moves in pace with the magnetic source to actively track the controlled element, wherein the eye-to-hand imaging system is controlled by a mobile structure to provide imaging and localization information for a patient body: (c) a robotic platform that consists of a ground rail that provides stable position adjustment of the magnetic source around the patient bed and a heavy load motion control mechanism that consists of a link-leverage structure and two linear actuators: (d) an integrated patient bed that is compatible with medical monitoring devices and provides the patient condition to the central process module: (e) a central process module composed of host computers and DAQ micro-controllers that process the feedback data from the imaging system and gives orders to motion control unit and current control unit; and (f) a human-machine interface that translates operators' command to the specific motor and current signals and sends the orders to the corresponding subsystems, wherein the detailed operational process and controlled element's conditions are visualized in the monitors.

In one embodiment, the motion control of the magnetic source of this invention is realized by a compound leverage structure that could allow multiple DOF and heavy load at same time, wherein a magnetic source is supported by a rotational joint which stands on the base joint such that the weight is transferred to the base instead of being supported by the rotational joint, wherein the rotational joint is the fulcrum, wherein the distal end of the leverage is actuated by a linear actuator that controls the in-plane rotational motion, wherein the linear actuator connecting the magnetic source control the distance between the magnetic field and the patient.

In one embodiment, the imaging system of this invention is a collaboration of the motion control, eye-in-hand imaging module and eye-to-hand (external) imaging module, wherein a cooperation motion control method is invented to avoid interference by the magnetic actuation method and collision, wherein the ye-in-hand tracking device includes but not limited to ultrasound imaging, photoacoustic imaging, magnetic sensor array, the camera and infrared imaging, wherein the eye-to-hand imaging tool includes but not limited to X-ray, fluoroscopy, fluorescence imaging and laser-induced fluorescence.

In one embodiment, the ground rail around the patient bed of this invention is to support and control the pose of the magnetic source, wherein the arrangement of the rail can be two separate individual ones or a U shape that could cover the two ends of the patient bed, wherein the magnetic source number can be larger than two without influencing the imaging module and the patient.

In one embodiment, the electromagnetic source of this invention is controlled by an invented current control unit which consists of three units: bidirectional power supply units, h-bridge regulation units and micro-controller units.

In one embodiment, the magnetic controlled magnetic element of this invention includes a magnetic catheter, untethered magnetic robot, magnetic swarm particle and magnetic endoscope.

In one embodiment, the human-machine interface of this invention is an integrated module that combines a joystick, a pedal, a steering wheel and a 3D glass wherein the voice control, posture control algorithm and augmented reality technology are embedded.

In one embodiment, the imaging and tracking device of this invention collects multiple dimensional information to the central processing process includes internal body information, absolute targe poses, temperature information, magnetic field information and section-view of the patient body, wherein the data are fused together by the central process system which generates desired magnetic field and controls the movement of the robotic structure.

This invention also provides a method of controlling the integrated mobile magnetic actuation and imaging system that could realize simultaneous localization and navigation of a magnetic element. In one embodiment, said method comprises: (a) imaging module provides the position information of the controlled element, the magnetic source position, the robotic platform structure to the central process system: (b) data fusion, desired magnetic field calculation, operation order translation and movement control of the robotic structure are processed on the central process system which sends the control signals to the distributed process unit of each subsystem: (c) the magnetic source is moved to the vicinity of the magnetic element and calculates the currents that could generate desired magnetic field and the temperature control unit controls the cooling and the circulating of the coolant in the internal and external cooling channel: (d) imaging tool motion control unit moves the external imaging device such that the magnetic element is moving in the field of view and the current position of the element is feedback to the central process system: (e) desired current and movement of the magnetic source are updated on the central process unit: (f) the human-machine interface updates the operation orders from the operator and translates the orders to the specific magnetic field and movement requirement to the center process system: (g) the patient's information, the magnetic element condition and internal information of the body are presented on the screen to assist the operation.

In one embodiment, the temperature control system of this invention consists of two units, wherein the internal circulating channels are embedded in the soft iron cores with temperature sensors installed at the inlets and outlets, wherein the external circulating channel is bounded on the surface of the electromagnetic coils with temperature sensors embedded on the core surface, the coils surface, the inlets and outlets of the circulating channel.

In one embodiment, the internal cooling system of this invention is designed to balance the total volume and the heat generation, wherein the channels are distributed through the core.

In one embodiment, a magnetic modeling method for the unconventional electromagnetic coil which has internal structured soft iron cores and customized electromagnetic coil shapes, wherein the simulated magnetic field is calibrated with the manually measured field by a learning-based modeling method.

In one embodiment, the eye-in-hand imaging system of this invention comprises one or more types of tracking tools such as ultrasound imaging, magnetic sensor array and cameras, whereas the ultrasound probe is controlled by a positioning device such as a Stewart platform or rotational joint.

This invention further provides a hierarchical control method to coordinate the motion control and magnetic field generation to avoid collision and interference between imaging system and actuation system, wherein the first level is the safety distance control and collision control, wherein the second level is field generation control and imaging field of view control.

In one embodiment, the controlled magnetic element includes but is not limited to the magnetic catheter, untethered magnetic microrobot, and magnetic capsule endoscope.

This invention further provides an electromagnetic shielding design which is made of multiple layers of material with different magnetic permeability, whereas the shape of the electromagnetic is designed to close the magnetic field inside the workspace.

This invention also provides a magnetic field monitoring system design that consists of different sensors including but not limited to magnetic sensors, temperature sensors, and humidity sensors; whereas the position of the sensors are positioned in a relatively large distance from the workspace and the real-time electromagnetic source status can be observed.

This invention provides a device for tracking of a magnetic element. In one embodiment, said device comprises: a) a magnetic actuation unit, comprising: i) an eye-in-hand sensing module: ii) a plurality of magnetic sources arranged evenly around said eye-in-hand sensing module, each of said plurality of magnetic sources is tilted at a tilt angle and separated from an adjacent magnetic source at an adjacent angle; and iii) an adjustment mechanism connected to each of said plurality of magnetic sources for adjusting said tilt angle and said adjacent angle to achieve a desired magnetic field: b) a robotic platform for mounting of said magnetic actuation unit and providing dexterous pose control of said magnetic actuation unit.

In one embodiment, said eye-in-hand sensing module comprises one or more components selected from the group consisting of cameras, 2D/3D ultrasound probes, x-ray generators, electrostatic sensors, magnetic sensors, fluorescent sensor, optical transmissometer, force sensors, grating sensor, photoacoustic probes, laser speckle imaging, infrared thermography cameras, radio frequency probes and humidity sensors. In another embodiment, said ultrasound probe is controlled by a positioning device selected from the group consisting of steward platform and rotational joint.

In one embodiment, said adjustment mechanism comprises a configuration selected from the group consisting of: a) two linear actuators (17) and two universal ball joints (19): b) a four-bar linkage formed by four rotational joints (26, 27): c) a linear actuator (28), a universal ball joint (29) and a rotational joint (30); and d) a linear actuator (31) or a rotational actuator.

In one embodiment, said plurality of magnetic sources comprises electromagnets or permanent magnets. In another embodiment, said electromagnets comprises a soft iron core, copper wire, and a temperature control module for controlling temperature of said soft iron core, said temperature control module comprises: a) a cooling channel having an inlet and an outlet, wherein said cooling channel is looped around or within said soft iron core: b) cooled heat-exchange fluid entering and leaving said cooling channel from said inlet and said outlet respectively; and c) temperature sensors installed at said inlet, said outlet and embedded within or on said soft iron core. In a further embodiment, said soft iron core has a stepped shape design. In yet another embodiment, said soft iron core comprises a tip shape that is cylindrical, cone, stepped or round.

In one embodiment, a current control unit is used for controlling electric current supplied to said electromagnets to achieve said desired magnetic field, said current control unit comprises bidirectional power supply units, h-bridge regulation units and micro-controller units.

In one embodiment, said magnetic element is selected from the group consisting of tethered magnetic robots, magnetic catheters, magnetic guidewires, magnetic needles, magnetic sleeves, magnetic soft robots, magnetic continuum robots, untethered magnetic robot, magnetic helical swimmers, magnetic rollers, magnetic grippers, magnetic surgical tools, magnetic endoscopes, and magnetic capsule endoscopes.

In one embodiment, said device further comprises a magnetic isolation shield made of layers of materials with different magnetic permeability.

In one embodiment, said robotic platform comprises a configuration for dexterous pose control of said magnetic actuation unit, said configuration is selected from the group consisting of: a) two rotational joints (36, 37) to control in-plane motion and two rotational joints (38) to control the out-of-plane motion: b) a first linear actuation mechanism (39) supported by a first rotational joint (40) and a second rotation joint (42), wherein said second rotation joint is connected to a second linear actuation mechanism (41); and c) a rotational actuator (47) and a linear actuator (48).

In one embodiment, said robotic platform comprises a rail (43) for stable translation motion of said device.

This invention also provides a system for tracking of a magnetic element. In one embodiment, said system comprises: a) a magnetic element: b) one or more devices of claim 1: c) an eye-to-hand imaging system (2); and d) a computer processor: wherein said computer processor executes an algorithm for collaborating said one or more devices and said eye-to-hand imaging system (2), said algorithm comprises the steps of: i) receiving pose information of components in said system: ii) calculating desired trajectories of said one or more devices of claim 1 and said eye-to-hand imaging system (2) based on a command for manipulating said magnetic element from a user before an operation: iii) analyzing real time positions of objects in field-of-view of said eye-to-hand imaging system during said operation; and iv) adjusting said desired trajectories based on said real-time positions using a predictive control strategy to detect potential collision.

In one embodiment, said system further comprises a patient bed embedded with a sensing and tracking module.

In one embodiment, said eye-to-hand imaging system is integrated with a mobile C-arm.

In one embodiment, said magnetic element is selected from the group consisting of magnetic catheter, untethered magnetic microrobot, and magnetic capsule endoscope.

In one embodiment, said plurality of magnetic sources comprises electromagnets and a current control unit for controlling electric current supplied to said electromagnets to achieve said desired magnetic field, said current control unit comprises bidirectional power supply units, h-bridge regulation units and micro-controller units.

In one embodiment, said algorithm further comprises a hierarchical control method to coordinate motion and magnetic field generation of said system, said hierarchical control method comprises a first level to control a safety distance and prevent collision: a second level to control magnetic field generation and imaging field of view.

In one embodiment, said system further comprises a module for a user to intervene and dominate control of said system over said algorithm.

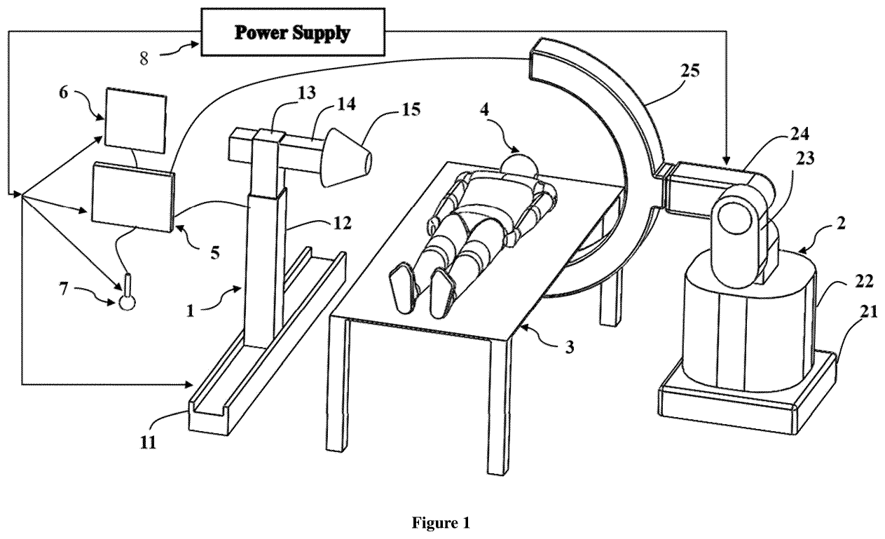

FIG. 1 shows a schematic view of an embodiment of the integrated magnetic actuation and navigation system. The said system comprises a mobile magnetic control device 1 with human-scale workspace, a. imagery equipment 2 and a support bed 3 for a patient 4, a central processing system 5, a visualization system 6, a human-machine interface 7, and a power control unit 8. According to the invention, the mobile electromagnetic source 1 is designed for actuating an element in the said patient's body 4. The mobility of the mobile is achieved by the robotic platform consisting of 11 12 13 14. 11 is a linear rail actuator that moves the magnetic source in a human-size range. 12 and 13 realize local magnetic source adjustment. The magnetic actuator mounted on 14 comprises a cluster of magnetic sources. In one embodiment, a set of electromagnets is utilized to generate a controllable magnetic field, such as a uniform field or gradient field. The external medical imaging subsystem moving mechanism 2 is designed to provide the human scale tracking and imaging ability. The said imaging system has a base joint 21, a rotational waist joint 22, two rotational joints 23, 24 and a C-shaped arm 25 at the end.

In the presented embodiment in FIG. 1, the central processing module 5 controls the motion of the said magnetic actuation subsystem 1 and the magnetic field generation. In one embodiment, the said central processing module consists of host computers and microcontrollers. In one embodiment, the pose control device is used to control the permanent magnet to generate the desired magnetic field. In one embodiment, the supplied current of the electromagnetic source is controlled to generate the desired magnetic field. The said processing module collects the sensing data and generates desired magnetic field. In the present embodiment, a joystick 7 is used for convenient operation for operators where the magnetic field control and motion control algorithms are embedded in the human-machine interface. Other embodiments of human-machine interface such as touch screen, keyboard, posture control, and voice control are also applicable to the present invention. The said system comprises a power supply unit 8 to provide power for the robotic platform and the magnetic source. The imaging device 2 has an eye-to-hand configuration that the mobile support structure allows multiple DOFs control of the imaging region.

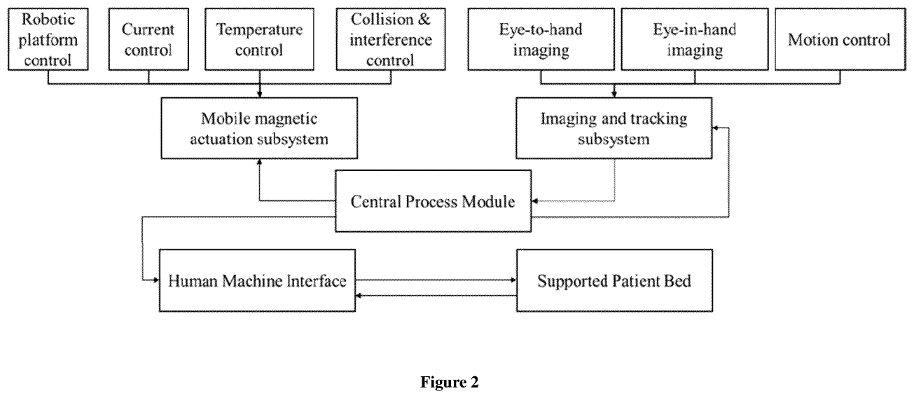

FIG. 2 presents the schematic drawing of the workflow of the integrated system. The whole system is made of five modules: 1) Mobile magnetic actuation subsystem that receives the command from the central process module and generates an accurate magnetic field in a large workspace, 2) imaging and tracking subsystem which provides pose information and the internal information of in the patient body, 3) a central process module that implements the control and tracking algorithm and coordinates the system to avoid interference and collision, 4) the human-machine interface which is convenient to access and could relief the working load of the physician, 5) the medical device supported patient bed that provides the patient's situations. In the mobile magnetic actuation subsystem, a robotic platform controls the magnetic source's pose and brings the actuation magnetic field to a human-scale workspace. In one embodiment, the magnetic source comprises a cluster of electromagnetic coils that generate desired magnetic field depending on the supplied current. The magnetic actuation subsystem translates the desired magnetic field to current and motion orders and sends this information to different modules. A temperature control module is integrated into the actuation subsystem to avoid overheating and nonlinearity of the field generation. Furthermore, the compatibility with the imaging and tracking subsystem is governed by the Collision & interference control module. In the imaging and tracking subsystem, the localization and imaging information from both the eye-to-hand and eye-in-hand imaging subsystems are fused to realize accurate in-body magnetic tracking and navigation of the controlled element. A motion control mechanism supports the eye-to-hand imaging and tracking system to realize large workspace imaging. In one embodiment, a mobile C-arm is adopted to locate the external imaging system. In one embodiment, a six DOF robotic arm is used to control the movement of the external imaging system.

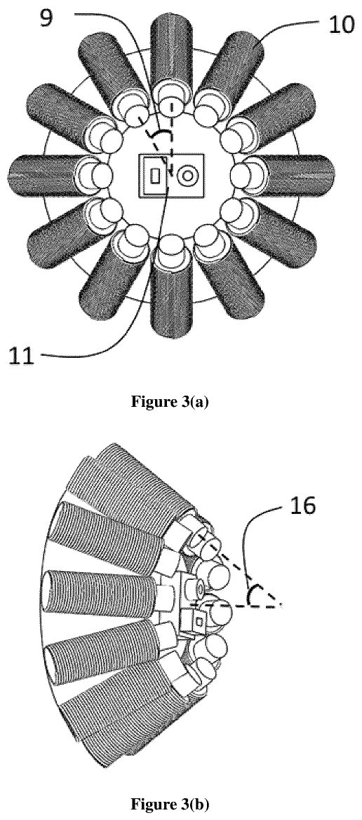

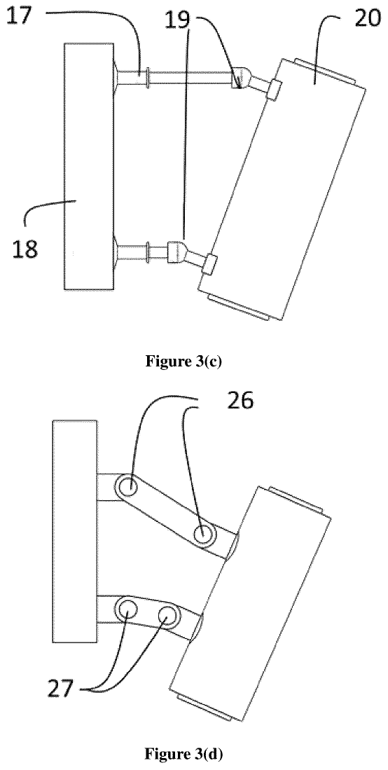

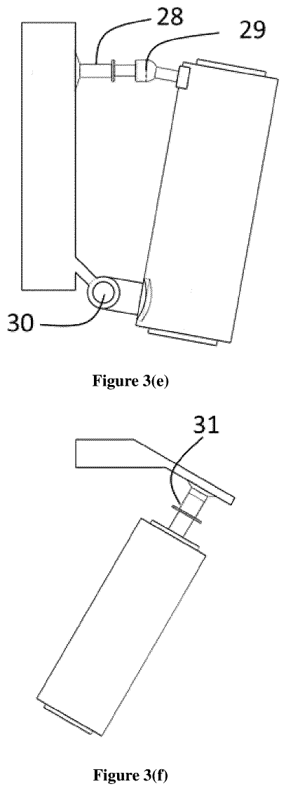

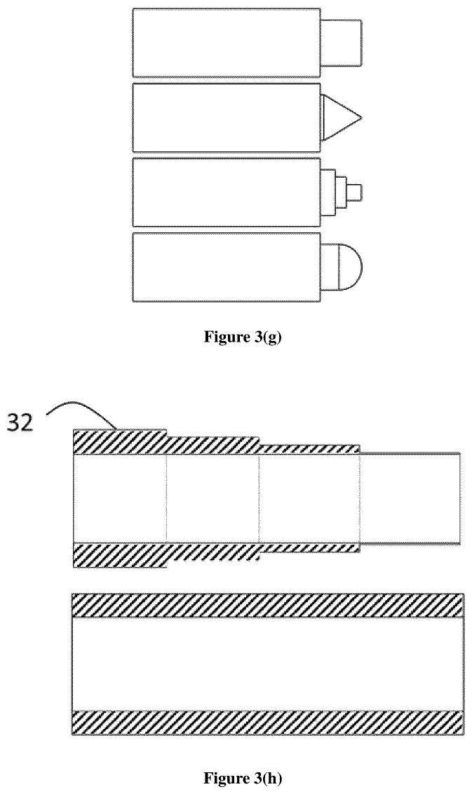

FIG. 3 shows the embodiments of several possible magnetic source designs from the invention. FIG. 3(a) shows one embodiment of the electromagnetic source design. The coils are evenly arranged around 11 the eye-in-hand sensing module for simple calculation. In one embodiment, ultrasound and camera are utilized to sense the controlled element. 9 is the adjacent angle between each coil's axis which determines the number of the electromagnetic coils. 10 is the individual magnetic coils composed of a soft iron core, copper wire, and cooling system. 16 is the tilt angle between the individual magnetic coil axis and the central axis. These two angles are designed according to the magnetic field requirement. The adjacent angle determines the individual magnetic source number and the DOF of the field generation. The tilt angle shapes the magnetic field generation profile. In one embodiment of the invention, the pose of an individual magnetic source can be configured by linear or rotational actuators. FIG. 3(c) shows the embodiment of the invention that shows the reconfigurable coils support structure design for an individual coil. 17 is the linear actuator, 19 are the universal ball joints. 18 is the static structure that connects to the robotic platform, 20 is the individual electromagnetic coil. FIG. 3(d) shows one embodiment of the invention that could adjust the pose of the individual electromagnet by one rotational actuator. 26 and 27 are rotational joints that form a four-bar linkage. The rotational actuator installed on one of the joints could change the pose of the magnetic source. FIG. 3(e) shows one embodiment of the invented pose adjustment device that is composed of a linear actuator 28, a universal ball joint 29, and a rotational joint 30. FIG. 3(f) shows the embodiment of the invented pose adjusting design that uses a single linear actuator to adjust the pose of the individual electromagnetic coil. FIG. 3(g) shows four embodiments of the invented core tip design. Cylindrical tip design allows a flattened magnetic field generation profile, and cone shape could concentrate larger field strength in the vicinity of the tip. Stepped shape tip design allows a stronger field in a relatively larger area in front. The round head design allows a magnetic field profile similar to the dipole model, which is convenient for field modeling. FIG. 3(h) shows two embodiments of the invented electromagnetic coils shape design. 32 is the stepped shape design of the coil shape that allows generating stronger field by the same amount of copper wire.

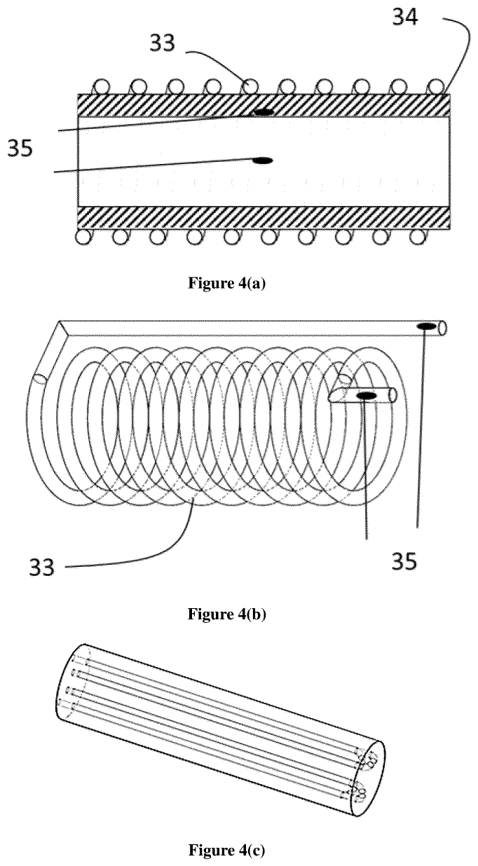

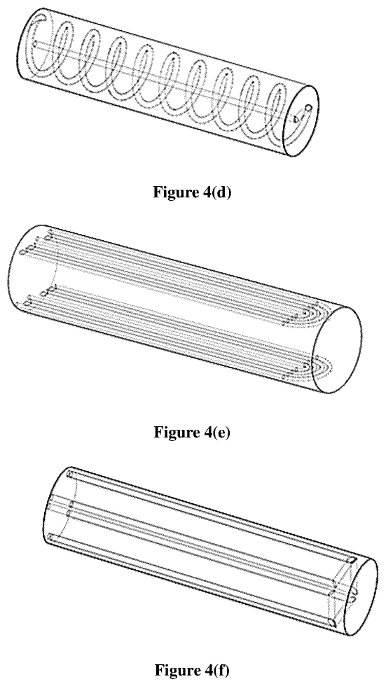

FIG. 4 shows the embodiment of the temperature control system design. FIG. 4(a) shows the cross-section of one embodiment of the electromagnetic coils cooling design. 33 is the cooling channel made of high heat conductive material such as copper. Cooled heat-exchange fluid is circulating in 33 to bring the extra heat from the surface of the electromagnetic coil. FIG. 4(b) shows one embodiment of the cooling channel design. Temperature sensors are installed in the soft iron core, inside the copper wire, the outlet and inlet of the circulation channel. FIG. 4(c-f) shows four embodiments of the invented core cooling system. The internal cooling channel of the soft iron core is circulating heat-exchange fluid same as the external cooling channel. Several sets of loops increase the cooling efficiency and the inlet and outlet temperature are monitored for circulation speed regulation. The embodiment of the channel shape design is presented in the figure. Because the cooling channel would occupy the volume of the soft iron core and thus reduce the magnetic field generation capacity, the cooling channel should be designed considering the cooling efficiency and the magnetic field generation requirements.

A magnetic field modeling method is invented for electromagnetic sources with the internal-structured soft iron core. Prior works reported the field modeling technique includes dipole field modeling, FEM modeling and Learning-based modeling. The invented hybrid modeling method is suitable for electromagnetic fields with various shapes and internal structures. There are three steps: firstly, a simulation of the magnetic field distribution of the CAD magnetic coil model is conducted. Secondly, magnetic distribution is manually measured at a number of points in the workspace or the region of interest. Thirdly, a learning-based mapping modeling method to calibrate the simulation data with the measured data. By increasing the number of measurements, we could increase the modeling accuracy:

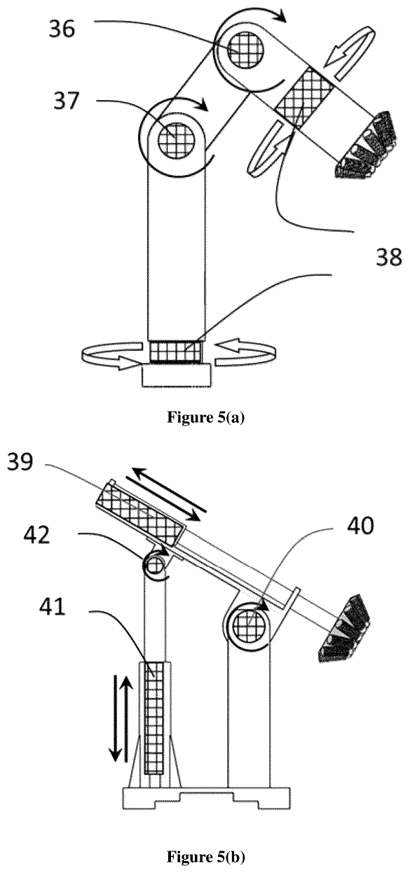

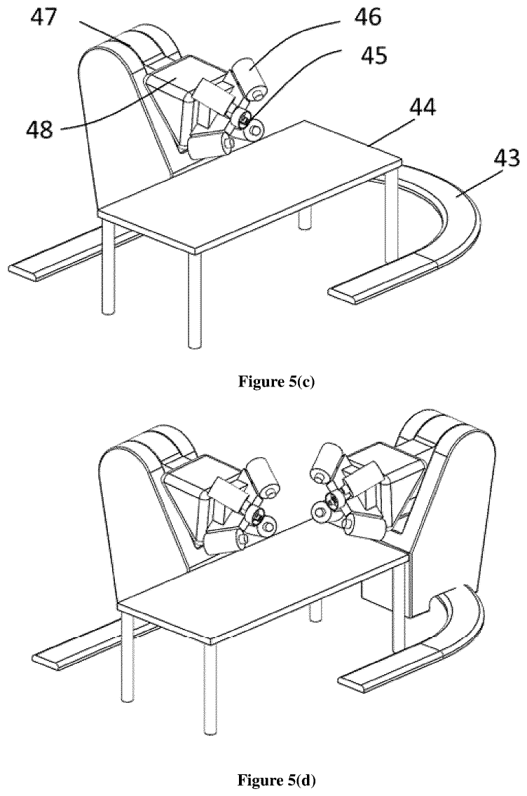

FIG. 5 shows the embodiment of the invented robotic platform design. The motion of the magnetic source is controlled by the robotic structure, which could provide dexterous pose control of the magnetic source. In one embodiment, as shown in FIG. 5(a), a serial robotic arm is designed to realize the motion control by using two rotational joints 36, 37 to control the in-plane motion and other two rotational joints to control the out-of-plane motion. Despite the limited load tolerance, the serial robotic arm could provide more degrees of freedom. In one embodiment, as shown in FIG. 5(b), the robotic platform is designed in a leverage structure for sufficient loading tolerance that could support a larger magnetic source. 40 is a passive rotational joint that supports a linear actuation mechanism 39. 41 is a linear actuator that can change the position of the rotational joint 42. The robotic structure could realize heavy load in-plane motion control by controlling the two actuators. FIG. 5(c) shows one embodiment of the invented robotic platform structure. 43 is the rail that is arranged around the patient bed, and this design allows stable translation motion of the robotic structure. The robotic structure is composed of rotational actuator 47 and linear actuator 48. 46 is one embodiment of the invented electromagnetic source. 45 is the integrated eye-in-hand imaging tool. FIG. 5(d) shows the embodiment of the invented design that uses mobile magnetic sources to realize multiple points of magnetic field control. The number of sets is determined by the magnetic field generation requirement and the space limitation.

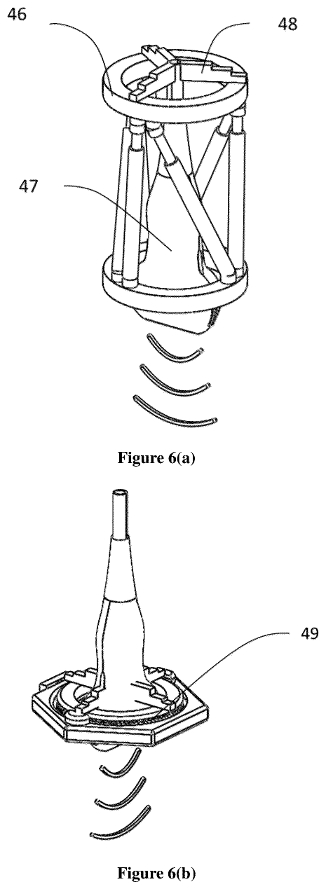

FIG. 6 shows embodiments of the eye-in-hand tracking system design. In the embodiment presented in FIG. 6(a), the ultrasound probe is held by a micro-Stewart Platform that could control 6 DOF of the US imaging plane. In one embodiment shown in FIG. 6(b), the one rotational DOF is controlled by a rotational actuator. It has a simpler design and could provide 2D in-plane information of the controlled target. The eye-in-hand imaging system would follow the movement of the controlled elements and provide effective and efficient localization.

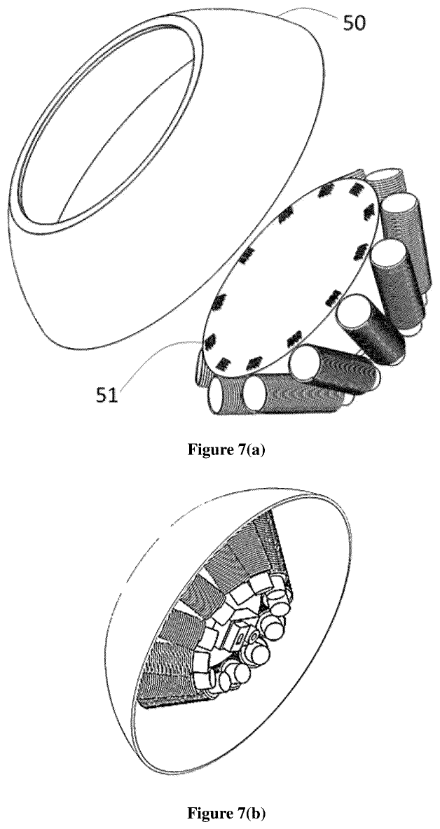

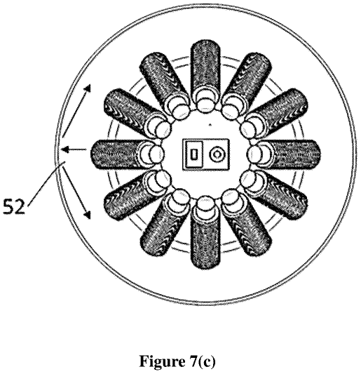

FIG. 7 shows embodiments of the magnetic isolation shield design 50 and the magnetic field monitoring system design 51. The shield is made of multiple layers that include high and low magnetic permeability material for isolating high and low frequency magnetic fields. Its shape is designed to close the magnetic field flux and restrict the magnetic field influence inside the workspace 52. The monitoring module is made of multiple sensors, including but not limited to magnetic, temperature, and humidity. Based on the extra information of the electromagnetic sources provided by the monitoring modules, the host computer could estimate the desired actuation field with higher precision.

The invention will be better understood by reference to the example details which follow, but those skilled in the art will readily appreciate that the specific structure described is only for illustrative purpose and are not meant to limit the invention as described herein, which is defined by the claims that follow thereafter.

Throughout this application, various references or publications are cited. Disclosures of these references or publications in their entirety are hereby incorporated by reference into this application to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example

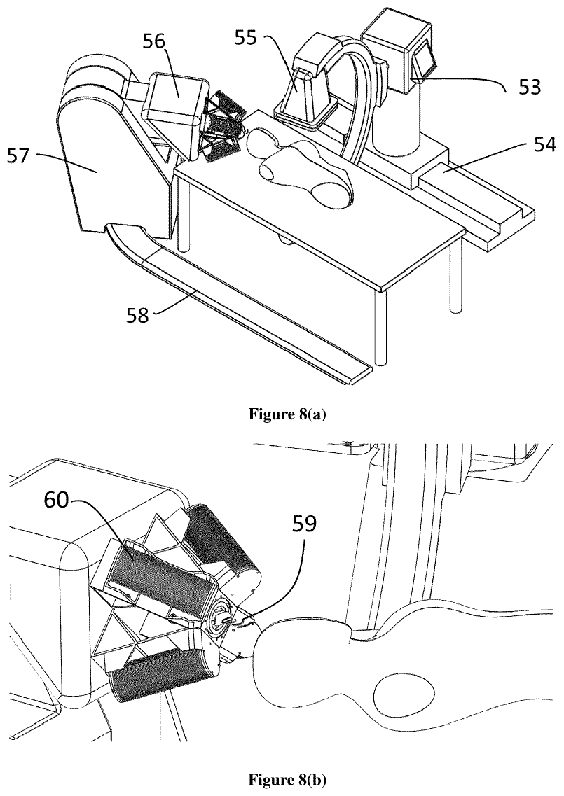

FIG. 8 shows an example design from the present invention that comprising four parts: a mobile magnetic source with 3-DOF motion control 56, 57, a C-arm supported external fluoroscopy that could adjust position and rotational angle 55, an eye-in-hand ultrasound tracking probe that provides accurate localization of the controlled object 59 and the central process system that could coordinate the whole system to realize in vivo magnetic actuation and tracking of the controlled object 53. FIG. 8(a) shows the arrangement of the subsystems such that the magnetic actuation and imaging tools share the same human-size workspace. The subsystems sit on rails 58. 54 that allow stable ground motion around the patient bed. FIG. 8(b) shows the details of the magnetic source design from the invention that consists of 4 coils and an eye-in-hand ultrasound imaging tool 59.

1-20. (canceled)

21. A device for tracking of a magnetic element, comprising:
a. a magnetic actuation unit, comprising:
i. an eye-in-hand sensing module;
ii. a plurality of magnetic sources arranged evenly around said eye-in-hand sensing module, each of said plurality of magnetic sources is tilted at a tilt angle and separated from an adjacent magnetic source at an adjacent angle; and
iii. an adjustment mechanism connected to each of said plurality of magnetic sources for adjusting said tilt angle and said adjacent angle to achieve a desired magnetic field;
b. a robotic platform for mounting of said magnetic actuation unit and providing dexterous pose control of said magnetic actuation unit;
wherein said robotic platform comprises a configuration for dexterous pose control of said magnetic actuation unit, said configuration is selected from the group consisting of:
a. two rotational joints (36, 37) to control in-plane motion and two rotational joints (38) to control the out-of-plane motion;
b. a first linear actuation mechanism (39) supported by a first rotational joint (40) and a second rotation joint (42), wherein said second rotation joint is connected to a second linear actuation mechanism (41); and
c. a rotational actuator (47) and a linear actuator (48).

22. The device of claim 21, wherein said eye-in-hand sensing module comprises one or more components selected from the group consisting of cameras, 2D/3D ultrasound probes, x-ray generators, electrostatic sensors, magnetic sensors, fluorescent sensor, optical transmissometer, force sensors, grating sensor, photoacoustic probes, laser speckle imaging, infrared thermography cameras, radio frequency probes and humidity sensors.

23. The device of claim 22, wherein said ultrasound probe is controlled by a positioning device selected from the group consisting of steward platform and rotational joint.

24. The device of claim 21, wherein said adjustment mechanism comprises a configuration selected from the group consisting of:
a. two linear actuators (17) and two universal ball joints (19);
b. a four-bar linkage formed by four rotational joints (26, 27);
c. a linear actuator (28), a universal ball joint (29) and a rotational joint (30); and
d. a linear actuator (31) or a rotational actuator.

25. The device of claim 21, wherein said plurality of magnetic sources comprises electromagnets or permanent magnets.

26. The device of claim 25, wherein said electromagnets comprises a soft iron core, copper wire, and a temperature control module for controlling temperature of said soft iron core, said temperature control module comprises:
a. a cooling channel having an inlet and an outlet, wherein said cooling channel is looped around or within said soft iron core;
b. cooled heat-exchange fluid entering and leaving said cooling channel from said inlet and said outlet respectively; and
c. temperature sensors installed at said inlet, said outlet and embedded within or on said soft iron core.

27. The device of claim 26, wherein said soft iron core has a stepped shape design.

28. The device of claim 26, wherein said soft iron core comprises a tip shape that is cylindrical, cone, stepped or round.

29. The device of claim 25, wherein a current control unit is used for controlling electric current supplied to said electromagnets to achieve said desired magnetic field, said current control unit comprises bidirectional power supply units, h-bridge regulation units and micro-controller units.

30. The device of claim 21, wherein said magnetic element is selected from the group consisting of tethered magnetic robots, magnetic catheters, magnetic guidewires, magnetic needles, magnetic sleeves, magnetic soft robots, magnetic continuum robots, untethered magnetic robot, magnetic helical swimmers, magnetic rollers, magnetic grippers, magnetic surgical tools, magnetic endoscopes, and magnetic capsule endoscopes.

31. The device of claim 21, further comprises a magnetic isolation shield made of layers of materials with different magnetic permeability.

32. The device of claim 21, wherein said robotic platform comprises a rail (43) for stable translation motion of said device.

33. A system, comprising
a. a magnetic element;
b. one or more devices of claim 21;
c. an eye-to-hand imaging system (2); and
d. a computer processor;
wherein said computer processor executes an algorithm for collaborating said one or more devices and said eye-to-hand imaging system (2), said algorithm comprises the steps of:
i. receiving pose information of components in said system;
ii. calculating desired trajectories of said one or more devices of claim 21 and said eye-to-hand imaging system (2) based on a command for manipulating said magnetic element from a user before an operation;
iii. analyzing real time positions of objects in field-of-view of said eye-to-hand imaging system during said operation; and
iv. adjusting said desired trajectories based on said real-time positions using a predictive control strategy to detect potential collision.

34. The system of claim 33, further comprises a patient bed embedded with a sensing and tracking module.

35. The system of claim 33, wherein said eye-to-hand imaging system is integrated with a mobile C-arm.

36. The system of claim 33, wherein said magnetic element is selected from the group consisting of magnetic catheter, untethered magnetic microrobot, and magnetic capsule endoscope.

37. The system of claim 33, wherein said plurality of magnetic sources comprises electromagnets and a current control unit for controlling electric current supplied to said electromagnets to achieve said desired magnetic field, said current control unit comprises bidirectional power supply units, h-bridge regulation units and micro-controller units.

38. The system of claim 33, wherein said algorithm further comprises a hierarchical control method to coordinate motion and magnetic field generation of said system, said hierarchical control method comprises a first level to control a safety distance and prevent collision; a second level to control magnetic field generation and imaging field of view.

39. The system of claim 33, wherein said system further comprises a module for a user to intervene and dominate control of said system over said algorithm.
